(19) United States
(12) Patent Application Publication
(10) Pub. No.: US 2011/0112436 A1
(43) Pub. Date: May 12, 2011

(54) Distraction pins for fluid aspiration

(75) Inventors: Robert J. Jones, Cedar Park, TX (US); Richard Kana, Lexington, TX (US); John B. Rossman, Austin, TX (US)

(73) Assignee: SpineSmith Partners, LP

(21) Appl. No.: 12/927,158

(22) Filed: Nov. 8, 2010

Related U.S. Application Data
(60) Provisional application No. 61/258,994, filed on Nov. 6, 2009.

(51) Int. Cl. A61B 10/00; B23P 15/52

(52) U.S. Cl. 600/567; 29/527.1

(57) ABSTRACT

A distraction pin for aspiration of a bone fluid and marrow. The pin includes an elongate metallic shaft of generally uniform diameter having a proximate end. The proximate end of the shaft includes an adaptor (attachment means) and a longitudinal bore extending from the distal end to the proximate end. The distal end of the shaft includes threading adapted for screwing the shaft into bone. The distal end also includes at least two fenestrations transverse to the longitudinal bore.

This application claims priority to U.S. Provisional Application Ser. No. 61/258,994, filed Nov. 6, 2009, which is incorporated in its entirety herein by reference.

### BACKGROUND OF INVENTION

This invention pertains to distraction pins capable of fluid aspiration.

During a typical spinal surgery, two or more caspar pins are screwed into vertebral bodies. A retractor, or similar instrument, is then attached to two or more caspar pins, allowing a surgeon to distract the vertebral bodies to accommodate a surgical procedure (e.g. spinal fusion, disc replacement, etc.). Distraction pins are typically used in cervical procedures, but may also be used in other lumbar or orthopedic procedures.

Recent studies have shown that there are a high percentage of stem cells present in the bone marrow in the cervical vertebral bodies of the spine. However, in order to remove this bone marrow, a surgeon must create a separate opening through the vertebral body and use a syringe or other aspiration device to extract bone marrow. This process increases the procedure time and subjects the patient through more risk.

### SUMMARY OF INVENTION

This invention provides a solution to one or more of the problems and/or deficiencies described in the background.

In one broad respect, this invention is a distraction pin, comprising an elongate metallic shaft of generally uniform diameter having a proximate end and a distal end, the proximate end including an adaptor (attachment means), the shaft including a longitudinal bore extending from the distal end to the proximate end, the distal end including threading adapted for screwing the shaft into bone, the distal end including at least two fenestrations transverse to the longitudinal bore. The distraction pin is adapted for aspiration of a bone fluid and marrow.

In another broad respect, this invention is a method of fluid aspiration, comprising:
providing a distraction pin comprises an elongate metallic shaft of generally uniform diameter having a proximate end and a distal end, the proximate end including an adaptor, the shaft including a longitudinal bore extending from the distal end to the proximate end, the distal end including threading adapted for screwing the shaft into bone, the distal end including at least two fenestrations transverse to the longitudinal bore;
attaching a distraction pin driving tool to the adaptor to allow the distraction pin to be screwed;
providing a bone having an outer layer and an inner area including marrow and fluid;
screwing the distraction pin into a bone so that the at least two fenestrations are enter the inner area;
detaching the distraction pin driving tool and aspirating marrow and/or fluid from the bone through the fenestrations and longitudinal bore.

In one embodiment, a distraction pin is inserted into a vertebral body with any known surgical technique. A distraction pin adapter is attached to the distraction pin, allowing the surgeon to use a retractor, or similar instrument, to distract the vertical bodies in preparation for a surgical procedure. When the distraction pin adapter is detached from the distraction pin, a fluid retrieval system may be attached to the proximal end of the distraction pin to remove fluid through a channel disposed through the distraction pin.

In another broad respect, this invention is a process for the manufacture of a distraction pin, comprising: forming an elongate metallic shaft of generally uniform diameter having a proximate end and a distal end, the proximate end including an attachment means, the shaft including a longitudinal bore extending from the distal end to the proximate end, the distal end including threading adapted for screwing the shaft into bone, the distal end including at least two fenestrations transverse to the longitudinal bore. The forming process can be made by injection molding, stamping, machining, and combinations thereof.

In another broad respect, this invention is a kit for removing marrow and/or fluid from a bone, comprising: (a) a distraction pin which comprises an elongate metallic shaft of generally uniform diameter having a proximate end and a distal end, the proximate end including an adaptor, the shaft including a longitudinal bore extending from the distal end to the proximate end, the distal end including threading adapted for screwing the shaft into bone, the distal end including at least two fenestrations transverse to the longitudinal bore; (b) a distraction pin driving tool adapted to connect to the attachment means; and (c) an aspiration tool adapted to connect to the attachment means.

### BRIEF DESCRIPTION OF THE FIGURES

FIG. 1 illustrates a cross-sectional view of a distraction pin of this invention.

FIG. 2 illustrates a detailed cross-section of section B of the distraction pin shown in FIG. 1.

FIG. 3 illustrates a side view of a distraction pin of this invention.

FIG. 4 illustrates a perspective detailed view of the threading and flutes of the distraction pin of FIG. 1.

FIG. 5 illustrates an exploded perspective view of the distraction pin, snap-ring, and driving tool.

FIG. 6 illustrates a detailed side and partially cross-sectional view of the driving tool.

### DETAILED DESCRIPTION OF THE INVENTION

Distraction pins may be used to facilitate distraction and deliver/aspirate fluid. Each distraction pin includes a channel disposed through the distraction pin shaft such that the channel is in fluid communication with the bone marrow. The channel facilitates the extraction or delivery of fluids, including bone marrow, through the distraction pin. The channel diameter may vary as a function of the concentration of fluid to be obtained or delivered.

The distraction pin may include other features, such as threading to stabilize the distraction pin, or fenestrations to facilitate the extraction or delivery of fluids. Fenestration holes may be strategically placed to localize where the fluid is drawn from and to avoid drawing peripheral blood from higher positions within the bone and from outside the cortical shell.

Cutting flutes may also be placed at the distal end of the distraction pin. These cutting flutes may be strategically placed to prevent inflow of peripheral blood that may dilute the stem cell concentration in the bone marrow.

The distraction pin may also include mechanisms to secure a fluid retrieval system or other surgical devices to the distraction pin, such mechanisms include a luer lock, collet, or other adapters. One example of a fluid retrieval system is a syringe with an aspiration needle. The aspiration needle may be sequentially pushed further into the bone to obtain various bone marrow samples from different depths of the vertebral body. Studies have shown that stem cell concentrations vary depending on the depth at which bone marrow is extracted.

A distraction pin may be driven into a vertebral body using any known surgical technique. For example, a driver may be attached to the proximal end of the distraction pin and used to drive the distraction pin into a vertebral body. The driver may include threading for attachment to a luer lock on the proximal end of the distraction pin. A snap ring may also be present to facilitate temporary attachment between the driver and the distraction pin.

If the driver attaches to a distraction pin using a luer lock, the driver may also include a locking mechanism to facilitate later removal of the distraction pin without unscrewing the luer lock attachment. Alternatively, a second driver with reverse threading may be used to remove the distraction pin when the procedure is complete.

The driver may incorporate attachment mechanisms other than a luer lock. For example, the driver may include retention fingers at the distal end of the driver. The retention fingers attach to retainer dimples on the proximal end of a distraction pin.

Distraction pins may vary in material and dimensions. The distraction pins may be constructed from stainless steel, a high strength industry-grade biocompatible polymer, or other materials. The diameter of the pin may vary as a function of the concentration or quantity of fluid to be obtained. The length of the distraction pins may also vary depending on whether the distraction pins will be used in conjunction with distraction pin adapters. Distraction pin dimensions may also vary in order to achieve a sturdy distraction pin or optimal torque on the shaft. Choice of material may also alter the dimensions of the distraction pin. The distraction pin may also take on various geometries. In a preferred embodiment, the distraction pin has a vertical shaft, but the distraction pin may take on other geometries.

In another embodiment, a channel is disposed through the distraction adapter such that the distraction pin adapter channel is in fluid communication with the distraction pin channel. This design permits the surgeon to simultaneously distract the vertebral bodies and aspirate bone marrow. After the distraction pins are inserted, the distraction pin adapters are attached to the distraction pins. The surgeon may use a retractor, or other similar instrument, in conjunction with the distraction pin adapters to separate the vertical bodies. At the same time, the surgeon may attach a fluid retrieval system to the proximal end of a distraction pin adapter to extract fluids.

In another embodiment, the distraction pin includes a longer shaft, such that the proximal end of the distraction pin extends out of the skin. This precludes any need for a separate distraction pin adapter and allows for easier attachment of larger syringes. The length of the shaft is at least long enough for the surgeon to use a retractor, or other similar instrument, without any need for the distraction pin adapters. A channel extends through the entire length of the distraction pin shaft such that the channel is in fluid communication with the patient's bone marrow.

In another embodiment, the proximal portion of the distraction pin takes on a curved configuration, such that the proximal portion of the distraction pin is not obstructing the surgical procedure. The proximal portion of the distraction pin is still designed for use with a retractor or other similar instrument. The distraction pin may include a channel disposed within the distal uncurved portion of the distraction pin. The channel is positioned such that it is in fluid communication with the patient's bone marrow. A port may be positioned proximal to the uncurved portion of the distraction pin to provide the surgeon with access to the channel. This design permits the surgeon to simultaneously distract the vertebral bodies and aspirate bone marrow, and increases the viewing area of the surgical field.

FIG. 1 shows a distraction pin 10 illustrative of this invention. The distraction pin 10 includes an elongate shaft 12. The distal end 14 includes a (conical) pointed tip 15. In this embodiment, the tip 15 does not include a cannulation or fenestration. The overall length of the pin can vary depending on the intended use and type of bone. Preferably the shaft length is such that that the proximate end extends out of the skin during use. For example, the pin 10 in FIG. 1 is approximately 3.4 cm., though the length can vary from about 2 to about 10 cm. The diameter o the shaft can also vary, such as from 2 to 10 mm. In one embodiment the diameter is about 5 mm.

The proximate end 16 of the pin 10 includes an adaptor 17. The adaptor 17 in this embodiment includes a hex head that 17A that is configured to mate with a socket of a tool (not shown in FIG. 1) that permits a surgeon to drive/screw the distraction pin into a bone. The adaptor 17 in FIG. 1 also includes a section a luer lock 17B for attaching an aspiration device (such as a syringe). The luer lock 17B is typically smaller in diameter than the hex head 17A to permit the driving tool to attach over the luer fitting. While in one embodiment the adaptor is monolithic with the shaft or otherwise permanently mounted to the shaft, the adaptor may alternatively be detachable from the shaft such as through us of threading. FIG. 1 shows an adaptor bore 22 within the adaptor 17 whose void lies under both the hex head 17A and luer lock 17B.

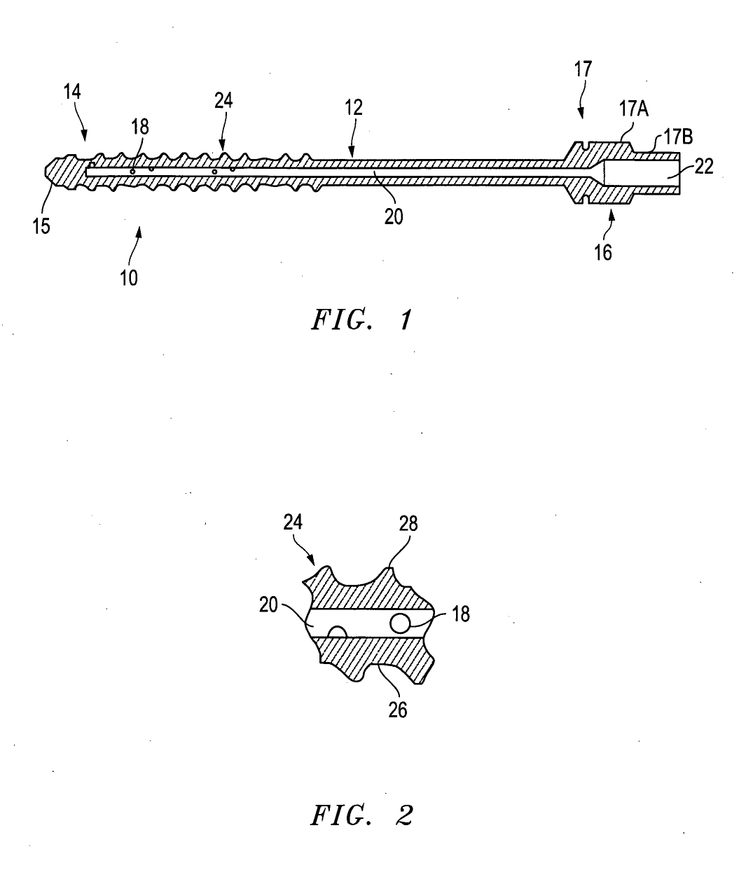

In FIG. 1, the distal portion of the pin 10 includes threading 24. That is, approximately one-half the length of the pin 10 is threaded. Alternatively, the shaft can be fully threaded. The threading can have the particular shape and size shown in FIG. 2, but other shapes and sizes can also be used. In pin 10 the shaft 12 and threading 24 both have uniform diameters over their lengths. Likewise, in FIG. 1 the outside diameter of the threading is slightly larger than the outside diameter of the shaft.

The shaft 12 includes a longitudinal bore 20 which is open at the proximal end 16 but falls short of the conical tip 15. In FIG. 1, the longitudinal bore terminates relative to the proximate end at roughly the second turn of threading 24. The bore, however, may terminate at other locations of the shaft. The bore 20 may be any diameter suitable for the intended use. In FIG. 1 the diameter of the longitudinal bore 20 is approximately one-third the diameter of the shaft 12. The diameter should not be so great as to jeopardize the structural integrity of the pin 10 for the intended use.

As depicted in FIG. 1 the shaft includes fenestrations 18, that is, holes, which are transverse and bisect the longitudinal bore 20. The fenestrations 18 sized and configured to serve as conduits for materials to flow from within a bone into the bore 20 and out into an aspiration device (not shown). The shape of the fenestrations are generally circular but can be oval or otherwise. The diameter of the fenestrations can vary. In the embodiment shown in FIG. 1, the fenestrations are about one quarter of the shaft diameter. As shown in FIG. 3, the fenestrations are generally positioned within the valleys 26 of threading, and are generally midway between a valley 26 and a ridge 28 of the threading 24.

FIG. 3 shows advantageous markings 30 which can be included in one embodiment of the invention. These markings 30 serve to inform the surgeon how deep the distraction pin 10 has been positioned within the bone at any given time.

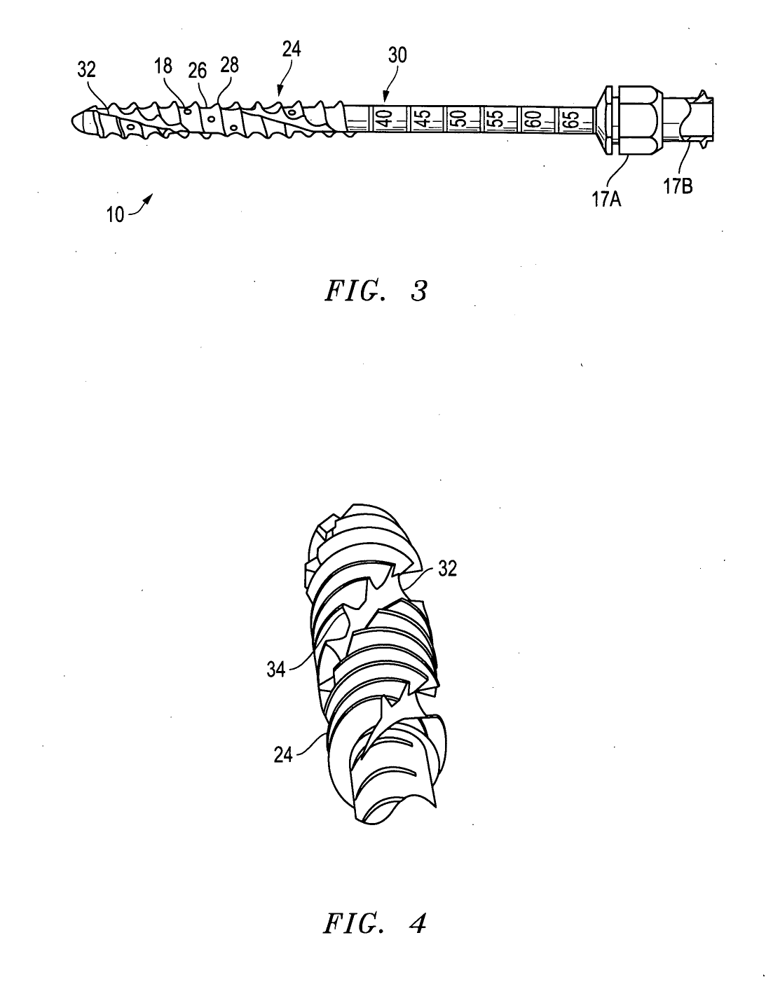

In the pin 10 depicted in FIG. 3, the threading is partially absent in serpentine fashion. This may be referred to as flutes 32 which can facilitate the advancement of the pin 10 as it is screwed into the bone. As shown in FIG. 4 a flute provides a cutting edge 34 on the threading 24. The flutes 32 may also serve to facilitate fluid aspiration into the fenestrations 18.

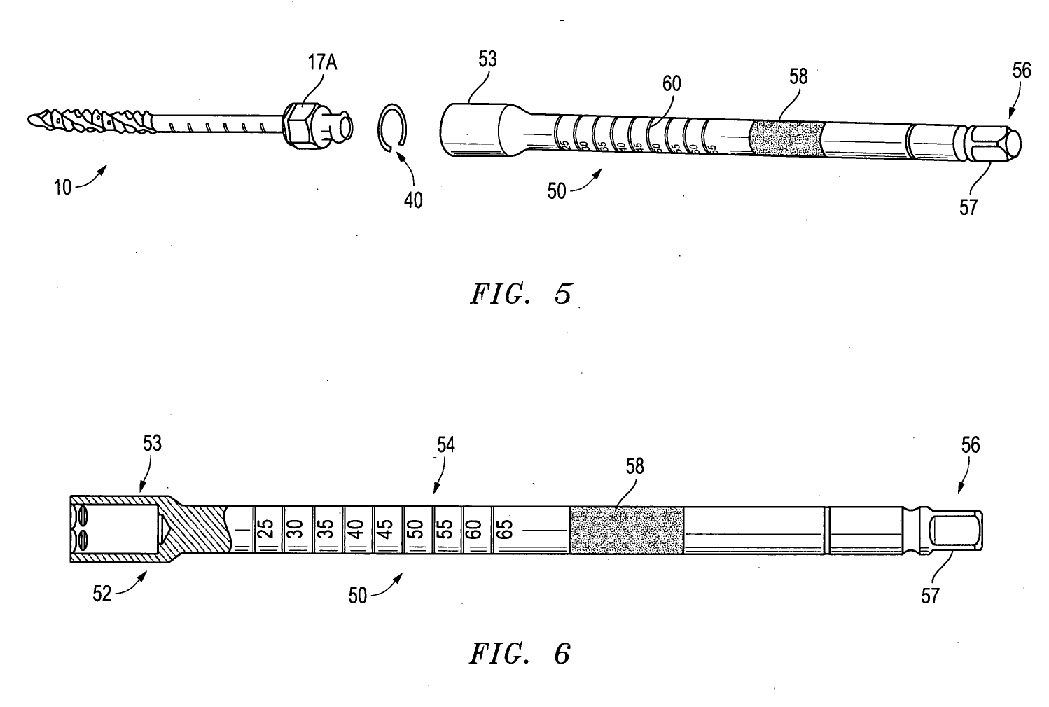

FIG. 5 illustrates a distraction pin with a complementary driver tool 50 and snap-ring 40 for distraction pin 10. The driver tool is shown in more detail in FIG. 6. The driver tool 50 has a distal end 52 and a proximate end 56 of tool shaft 54. The distal end 52 includes a hex socket head 53 that is configured and sized to fit the hex head 17A of distraction pin 10. The proximate end 56 includes a male socket 57 that is adapted to fit, for example, the chuck of a drill, or to fit a complementary bar (not shown) to form, for example, a T-handle for manual application of force to strew the distraction pin 10 in or out of bone. The shaft can also include markings 60 to assist a surgeon in determining how far the distraction pin 10 has advanced in a bone. The snap-ring 40 fits over the luer lock 17B, serving to lock the driving tool 50 in place. The shaft 54 can include a cross-hatched knurl 58 portion that aids in grasping the driving tool 50.

The distraction pin 10 and driving tool 50 can be formed of any suitable metal or alloy conventionally used in surgical equipment. The material should be tested to determine whether the pin will break or shear off during use. The tip of the distraction pin can be, for example, made of titanium, stainless steel, titanium-nickel, and so forth.

During use, the distraction pin is used in surgery to remove blood, marrow, fluid, and/or other materials from within bone. After the bone is accessed and optionally drilled to provide a starting hole or full bore for the distraction pin, the driving tool, snap-ring, and distraction tool are wedded together. The tip of the distraction tool is then placed at the point of the bone where material is to be aspirated. Then the driving tool is turned so that the distraction pin screws into the bone to a desired depth as shown by the shaft markings, for example. The snap-ring and driving tool are then separated from the distraction pin, and an aspiration tool such as a syringe is attached to the luer fitting of the distraction pin. Next, the aspiration tool is manipulated to extract fluid, blood, marrow, and/or other materials from the inside of the bone. Afterward, the aspiration tool is removed and the driving tool reattached to the distraction pin so that the distraction pin can be back out of the bone.

It is envisioned that the present invention may be used to address various conditions through use of the fibrin sealant in a manner similar to that described in the examples above. Discussion of this invention referenced particular means, materials and embodiments elaborating limited application of the claimed invention. The invention is not limited to these particulars and applies to all equivalents. Although this invention has been described above with reference to particular means, materials and embodiments, it is to be understood that the invention is not limited to these disclosed particulars, but extends instead to all equivalents within the scope of the following claims.

What is claimed is:

1. A distraction pin for aspiration of a bone fluid and marrow, comprising an elongate metallic shaft of generally uniform diameter having a proximate end and a distal end, the proximate end including an adaptor (attachment means), the shaft including a longitudinal bore extending from the distal end to the proximate end, the distal end including threading adapted for screwing the shaft into bone, the distal end including at least two fenestrations transverse to the longitudinal bore.

2. A method of fluid aspiration, comprising:
providing a distraction pin comprises an elongate metallic shaft of generally uniform diameter having a proximate end and a distal end, the proximate end including an adaptor, the shaft including a longitudinal bore extending from the distal end to the proximate end, the distal end including threading adapted for screwing the shaft into bone, the distal end including at least two fenestrations transverse to the longitudinal bore;
attaching a distraction pin driving tool to the adaptor to allow the distraction pin to be screwed;
providing a bone having an outer layer and an inner area including marrow and fluid;
screwing the distraction pin into a bone using the distraction pin driving tool so that the at least two fenestrations are enter the inner area;
detaching the distraction pin driving tool; and
aspirating marrow and/or fluid from the bone through the fenestrations and longitudinal bore.

3. A process for the manufacture of a distraction pin, comprising: forming an elongate metallic shaft of generally uniform diameter having a proximate end and a distal end, the proximate end including an adaptor, the shaft including a longitudinal bore extending from the distal end to the proximate end, the distal end including threading adapted for screwing the shaft into bone, the distal end including at least two fenestrations transverse to the longitudinal bore.

4. The process of claim 3, wherein the forming process is injection molding, stamping, machining, or a combination thereof.

5. A kit for removing marrow and/or fluid from a bone, comprising: (a) a distraction pin which comprises an elongate metallic shaft of generally uniform diameter having a proximate end and a distal end, the proximate end including an adaptor, the shaft including a longitudinal bore extending from the distal end to the proximate end, the distal end including threading adapted for screwing the shaft into bone, the distal end including at least two fenestrations transverse to the longitudinal bore; (b) a distraction pin driving tool configured to connect to the adaptor; and (c) an aspiration tool configured to connect to the adaptor.
